(19) United States
(12) Patent Application Publication
(10) Pub. No.: US 2016/0278954 A1
(43) Pub. Date: Sep. 29, 2016

(54) DEVICE FOR FIXING AN ENDOPROSTHESIS

(71) Applicants: Carlo PEPE, Modena (IT); Giuseppe TUSCANO, Modena (IT); CALVI TECONOLOGIE S.A.S. DI ING, ANDREA CALVI & C, Lomagna (IT)

(72) Inventors: Carlo PEPE, Modena (IT); Giuseppe TUSCANO, Modena (IT); Roberto CALVI, Lomagna (IT)

(21) Appl. No.: 14/778,156

(22) PCT Filed: Mar. 20, 2014
(86) PCT No.: PCT/IB2014/060006

§ 371 Date: Sep. 18, 2015

(30) Foreign Application Priority Data
Mar. 20, 2013 (IT) BO2013A000120

(51) Int. Cl. A61F 2/89; A61F 2/848; A61F 2/07; A61F 2/844

(52) U.S. Cl. CPC A61F 2/89; A61F 2/844; A61F 2/848; A61F 2/07; A61F 2002/8483

(57) ABSTRACT

Device for fixing an endoprosthesis (endo-graft) either directly to the aortic wall and/or at distal level to avoid migration of the endoprosthesis (endo-graft); the device comprises a crown of springs connected to one another adapted to exert an outwards radial spring-back force so as to push the endoprosthesis against the aortic wall and therefore maintain the endoprosthesis in position; in the area of the springs couplings are provided, adapted to penetrate the aortic wall.

### TECHNICAL FIELD

The present invention relates to a fixing device, a system for repairing a duct of the human body and a kit for repairing a duct of the human body.

In particular, the present invention relates to a device for fixing an endoprosthesis to a duct of the human body, a system for endovascular repair of an aneurysm or of a non-vascular duct of the human body and a kit for endovascular repair of an aneurysm or of a non-vascular duct of the human body.

### CONTEXT OF THE INVENTION

The technique of endovascular aneurysm repair (EVAR) spread rapidly after the first operation by Dr. Parodi and has now become widely accepted despite the long-term results which are not always good due to the frequent need for secondary operations.

The cause of these secondary operations is often the presence of anatomical variations (wide, short, angled aneurysm neck) or complications.

There is a close relation between the anatomical variations and the complications, therefore there are errors in terms of both recommendations for an operation and surgical technique.

Due to these problems, many patients may not be candidates for the EVAR procedure; at least 50% of the, potential market.

The problems that hinder the feasibility, efficiency and above all the duration of EVAR include endoleaks (type 1) and above all migration of the endoprosthesis, which is now a well-known complication of EVAR. The endoprosthesis is considered to have migrated when a movement of the endoprosthesis greater than 5-10 mm or any displacement of the endoprosthesis requiring a secondary correction occurs.

An endoleak is the infiltration of blood inside the aneurysm sac despite the presence of a prosthesis, the purpose of which is to exclude the aneurysm from the blood circulation.

A favourable result after EVAR depends on the persistent fixing of the endograft. For this reason, prostheses have been proposed with innovative design and the use of hooks and barbs etc.

The measures developed so far, however, are complex (in terms of structure and/or use) and/or not yet able to guarantee high level results.

US20020183833 describes an expandable frame comprising a plurality of flexible joints alternating with connecting arms.

EP839505 describes a self-expanding endoprosthesis.

U.S. Pat. No. 6,814,748 concerns an intraluminal grafting system comprising a self-expanding attachment system.

The object of the present invention is to provide a device for fixing an endoprosthesis, a system for repairing a duct of the human body and a kit for repairing a duct of the human body which overcome, at least partially, the drawbacks of the known art and at the same time are easy and inexpensive to produce.

### SUMMARY

According to the present invention, a device is provided for fixing an endoprosthesis, a system for repairing a duct of the human body and a kit for repairing a duct of the human body according to the following independent claims and, preferably, any one of the claims depending directly or indirectly on the independent claims.

### BRIEF DESCRIPTION OF THE FIGURES

The invention is described below with reference to the attached figures, which illustrate some non-limiting implementation examples thereof, in which:

FIGS. 1-3 are frontal views in different operating configurations of a device according to the present invention;

FIG. 4 is an overhead view of the device of FIGS. 1-3 in different operating configurations;

FIGS. 5 and 6 are frontal views of details of the device of FIGS. 1-3 in different operating configurations;

FIG. 7 is a perspective view partially in section of an example of utilisation of a device according to the present invention;

FIGS. 8-10 are lateral views of different embodiments of a detail of the device of FIG. 7;

FIGS. 11 and 12 are views from below of the details of FIGS. 5 and 6, respectively; and

FIG. 13 is a perspective view partially in section of a further example of use of a device according to the present invention.

### EMBODIMENTS OF THE INVENTION

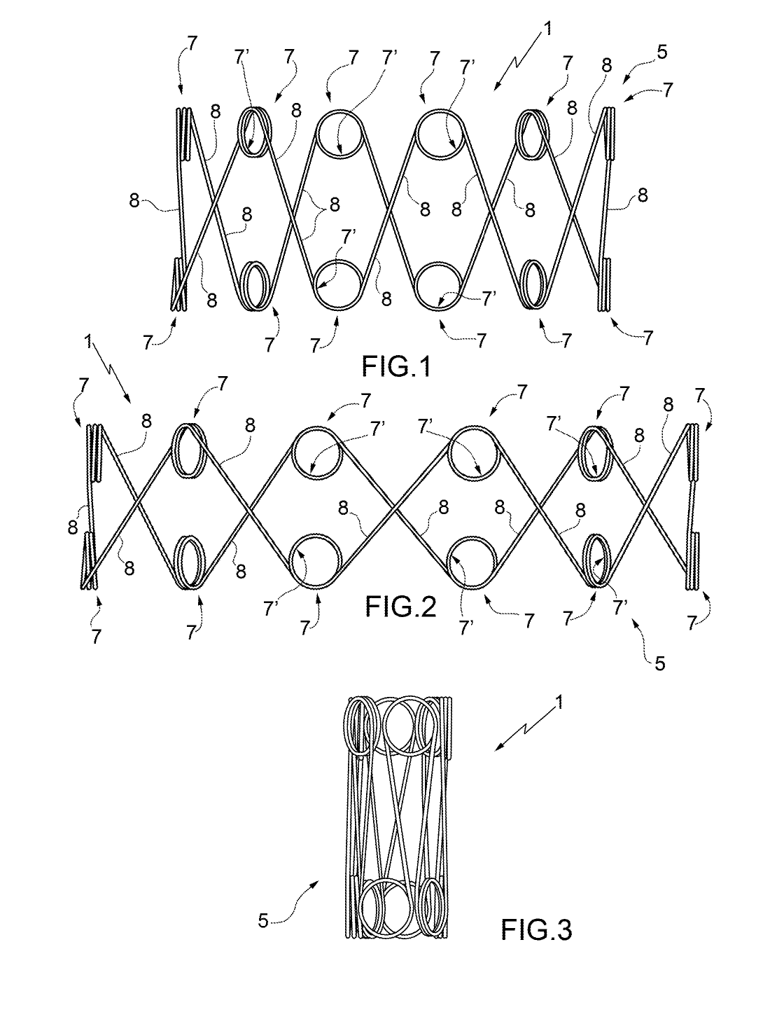

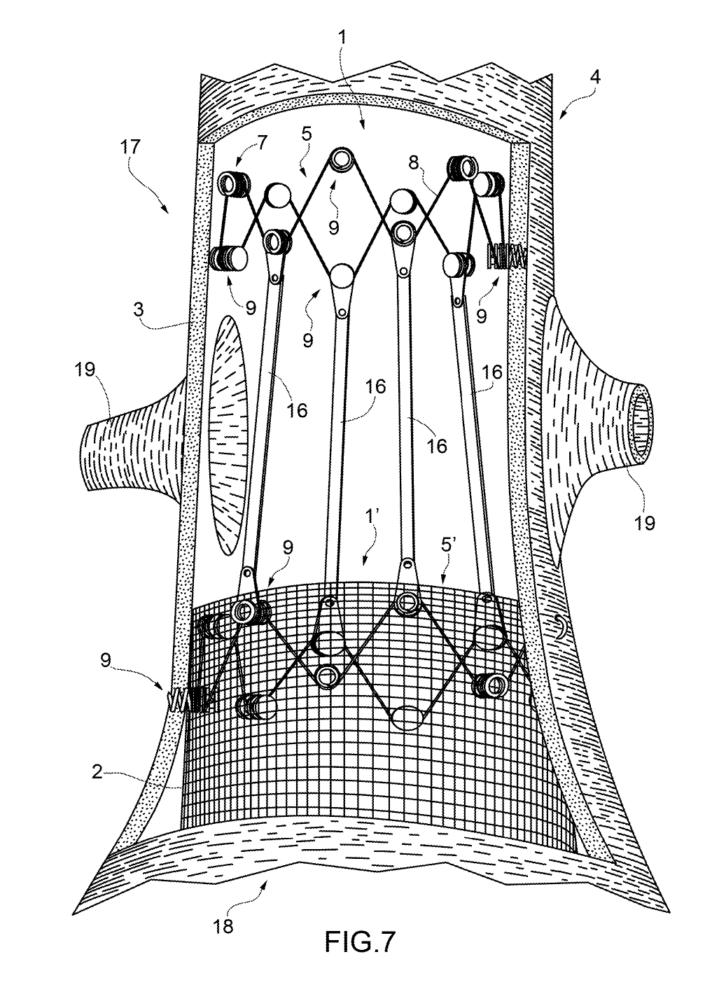

According to a first aspect of the present invention, in FIG. 1, number 1 indicates as a whole a device for fixing an endoprosthesis 2 (endo-graft) to a wall 3 (FIG. 7) of a blood vessel 4 (more precisely, an aorta).

Said endoprosthesis is fixed to an inner surface of the blood vessel 4.

According to other embodiments, the device 1 is adapted to be fixed (internally) to a wall of a non-vascular duct of the human body (for example oesophagus, trachea etc.).

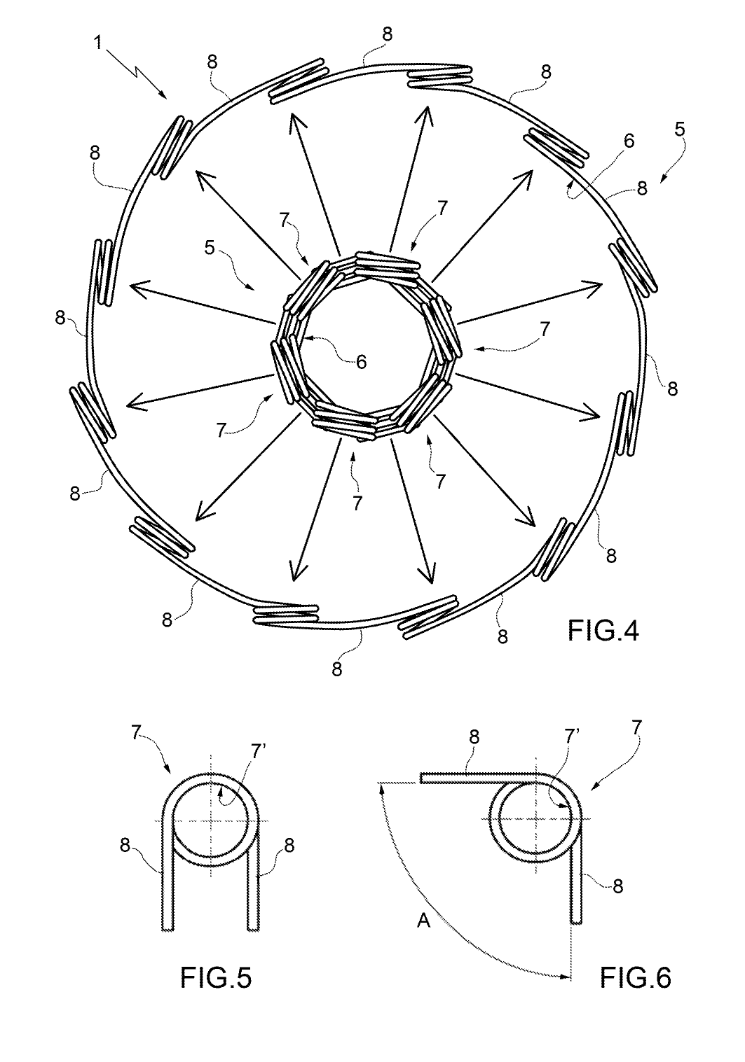

With reference to FIG. 4, the device 1 comprises a thrust element 5, which has the shape of a closed curve peripherally delimiting an inner free space 6 and is adapted to exert an outwards spring-back force. In particular, the thrust element 5 has a crown shape and, is adapted to exercise the spring-back force radially (in the direction of the arrows of FIG. 4).

The thrust element 5 comprises a plurality (in this case twelve) of springs 7 (in particular, torsion springs) connected to one another. Each spring 7 is connected integrally to two springs 7 so that the springs 7 are arranged in sequence. More precisely, a plurality of connecting arms 8 are provided to each connect two successive springs 7.

The arms 8 have an elongated shape, more precisely oblong. Advantageously (for the particular use proposed), the connecting arms 8 have a length ranging from approximately 5 mm to approximately 5 cm.

Each spring 7 comprises at least one loop (advantageously, at least two loops) and is structured so as to aid extension of the thrust element 5. In other Words, the particular structure of the springs 7 is such as to bring the thrust element 5 from a substantially packed configuration (or closed—as illustrated in FIG. 3 and in the inner part of FIG. 4) to a substantially extended configuration (or open—as illustrated in FIGS. 1 and 2 and in the inner part of FIG. 4). The loop/s of the spring 7 peripherally delimits/delimit an inner lumen 7′.

In other words, each spring .7 comprises a spring body (with one or more loops) and two arms 8, which project from the ends of the spring body. Each arm 8 is, therefore, shared by two successive springs 7.

In particular, each spring 7 comprises (more precisely, is) a cylindrical helical flexion spring (commonly known as torsion spring).

In actual use, the thrust element S is inserted into the blood vessel 4 (which has a lumen with radius smaller than the radius of the thrust element 5 at its maximum extension—FIG. 2) in its substantially packed configuration (FIG. 3). At this point, the thrust element 5 is extended (to the configuration illustrated in FIG. 2) so as to exert a force against the wall 3 to maintain the endoprosthesis 2 correctly in position.

It should be noted that the particular structure of the thrust element 5 allows excellent results to be obtained in terms of adhesion to the wall 3 independently of the specific form of the blood vessel 4. In this regard, it is important to note that, since the thrust element 5 may be extended to a greater or lesser extent (if necessary also differently in different sectors so as to take on a non completely circular configuration) it may adapt very well to different dimensions of the blood vessel 4 and to the imperfections (for example irregular shape) present on the inner surface of the blood vessel 4.

It is also important to consider that use of the device 1 is particularly simple since in one single operation (extension of the thrust element 5 inside the blood vessel 4) it is possible to stabilise the position of the endoprosthesis 2 inside the blood vessel 4.

Advantageously (for the particular use proposed), the inner free space 6 has an area ranging from 50 mm² (in particular, from 100 mm²) to 30 cm² (in particular, to 20 cm²) when the thrust element 5 is in the fully extended configuration (FIG. 2). The inner free space 6 has an area (from 1 mm²) up to 600 mm² (in particular, up to 201 mm²), when the thrust element 5 is in the packed configuration (FIG. 3).

Advantageously, each spring has a free angle A ranging from approximately 10° (in particular, from approximately 20°; more precisely, from approximately)30° to approximately 180° (in particular, to approximately 170°; more precisely to approximately 160°).

Referring in particular to FIGS. 5 and 6, by free angle A of a spring 7 we mean the angle between the connecting arms 8 to the spring 7 measured in the winding direction of the spring 7 (in particular of the loops of the spring 7), when the spring 7 is not subject to loads (or forces). FIG. 5 illustrates a free angle of approximately 20°, FIG. 6 illustrates a free angle of 90°.

Typically, when the thrust element 5 passes from the packed configuration to the extended configuration, the angle between the arms 8 of the same spring 7 is modified (increased) from 20° (in particular, from 30°) to 170° (in particular, to 150°).

With particular reference to the figures from 7 to 10, advantageously, the device 1 also comprises at least one coupling 9 (preferably at least two; in particular, at least three) which is adapted to fix the thrust element 5 to the wall 3. The coupling 9 is arranged in the area of a respective spring 7 (partially inside the lumen 7′).

According to the specific embodiment illustrated in FIG. 7, the device 1 comprises twelve couplings 9 (for each thrust element 5); in particular, one coupling 9 is provided for each spring 7.

The coupling 9 comprises a fixing element 10, which is adapted to penetrating the wall 3 and anchoring to the wall 3 itself (FIG. 7). In particular, the fixing element 10 is adapted to completely crossing the wall 10 (and anchoring to the wall 10 itself). Advantageously, the fixing element 10 is adapted to penetrating the wall 3 rotating around its own axis.

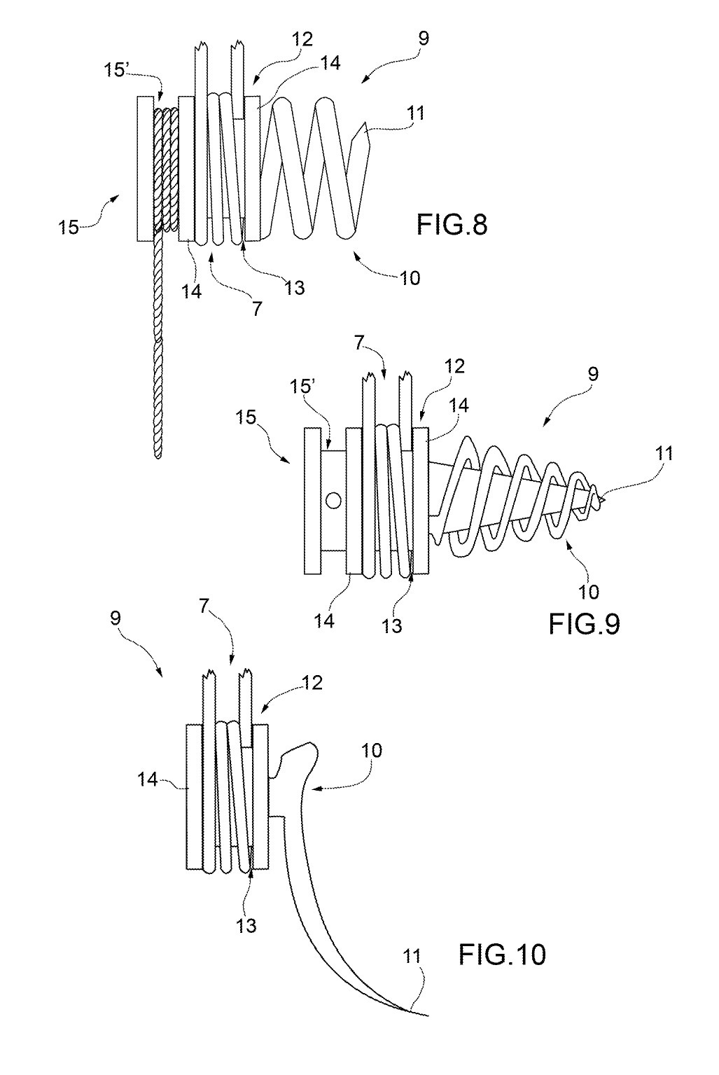

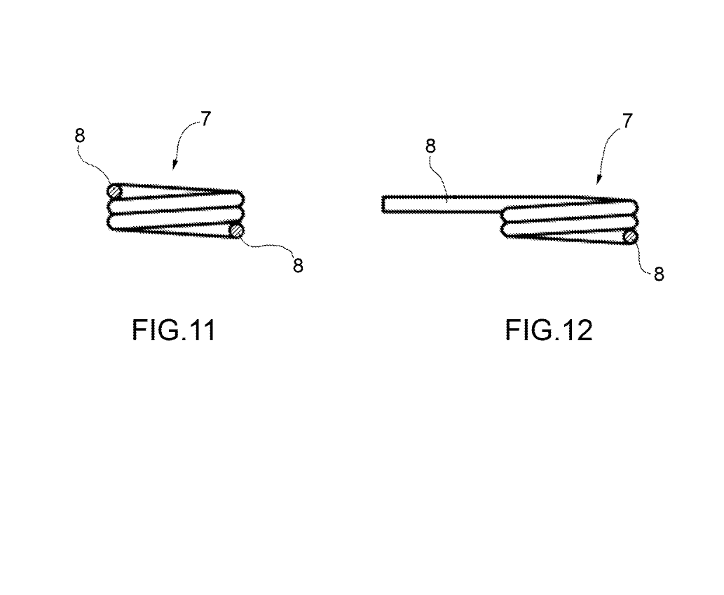

According to some variations, the fixing element 10 has a substantially helicoidal (as illustrated in FIG. 8), substantially spiral (as illustrated in FIG. 9) or hook shape (as illustrated in FIG. 10) (or a combination thereof).

In particular, FIG. 9 illustrates an embodiment in which the fixing element 10 has a double spiral shape. In other words, the fixing element (of FIG. 9) has a screw shape.

The fixing element 10 furthermore comprises a point 11 for perforating the wall 3 in the area of an end thereof (opposite the respective spring 7).

The coupling 9 also comprises a support portion 12, which is arranged inside the lumen 7′ (of the respective spring 7). The support portion 12 is provided with a seat 13, which houses the loop/s of the spring 7 and end walls 14 to maintain the loop/s of the spring 7 inside the seat 13.

The support portion 12 is integral with the fixing element 10 in the area of an end of :the fixing element 10 opposite the point 11.

The support portion 12 is structured so as to permit sliding of the loop/s inside the. seat 13 and relative rotation between the fixing element 10 and the spring 7. In particular, the seat 13 has a substantially annular shape.

Advantageously, the fixing element 10 has a longitudinal extension (from the support portion 12 to the point 11) ranging from 0.8 mm (in particular, from 1 mm) to 7 mm (in particular, to 5 mm).

According to some embodiments, the coupling 9 furthermore comprises a manipulation portion 15, which may be moved (in particular, rotated) to rotate the fixing element 10. The manipulation portion 15 is integral with the support portion 12 (on the opposite side with respect to the fixing element 10) and with the fixing element 10.

As illustrated in FIGS. 7 to 9, the manipulation portion 15 comprises an annular channel 15′, around which a thread is found (illustrated in FIG. 8).

In actual use, by pulling the thread the manipulation portion 15 is rotated, the latter transfers the movement to the support portion 12 and then to the fixing element 10. By rotating, the fixing element 10 perforates and penetrates the wall 3 so as to anchor to the wall 3.

It should be noted that the presence of the fixing elements further improves stabilisation of the endoprosthesis 2 inside the blood vessel 4. The rotatability of the fixing element 10 allows particularly simple and effective penetration and anchoring.

With particular reference to FIG. 7, according to some variations, the device 1 comprises at least two connecting elements 16 (advantageously, at least three; in particular, at least four) to connect the device 1 to the endoprosthesis 2. Preferably, the connecting elements 16 are arranged so as to be opposed (in pairs).

The connecting elements 16 have a variable length.

Each connecting element 16 is tied at one end to the thrust element 5 in the area of a respective spring 7. More precisely, the connecting elements 16 are tied to respective springs 7 arranged on the same side (above or below) of the thrust element 5.

In some cases the connecting element/s 16 are cables.

According to some embodiments, the device 1 comprises a further thrust element 5′ tied to the above-mentioned thrust element 5 by means of the connecting elements 16. The further thrust element 5′ is defined as descried above relative to the thrust element 5. Advantageously, hooks 9 are also provided associated (analogously to what is described for the thrust element 5) with the further thrust element 5′.

In other words, according to a second aspect of the present invention, a system 17 is provided for fixing an endoprosthesis 2 to the blood vessel 4 (or to a non-vascular duct of the human body). The system 17 comprises two devices 1 for fixing as described above to one another connected by means of two or more (advantageously, at least three; in particular, at least four) connecting elements 1 as described above.

According to a third aspect of the present invention, a system 18 is provided for the endovascular repair of an aneurysm (or non-vascular duct of the human body). The system 18 comprises at least one endoprosthesis 2 and at least one device 1 for fixing as described above (relative to the first embodiment of the present invention) tied to the endoprosthesis 2.

The device 1 is arranged in the area of one end of the endoprosthesis 2, in particular inside the lumen of the endoprosthesis 2. The couplings 9 (or the coupling 9) of the device 1 extend (or extends) partially through the endoprosthesis 2.

According to some embodiments, the system 18 comprises a further device 1′ for fixing as described above (relative to the first embodiment of the present invention) connected to the device 1 by means of two or more (advantageously, at least three; in particular, at least four) connecting elements 16 as described above. In these cases, in other words, the system 18 comprises the system 17 for fixing (as described above). In particular, the device 1′ is arranged in the area of the endoprosthesis 2 (in particular, of one end thereof), the other device 1 is arranged at a distance from the endoprosthesis 2.

According to a specific use of these embodiments of the system 18, the device 1 is arranged inside the aorta 4 above the renal artery 19 (in a distal position) and the device 1′ (the one arranged in the area of the endoprosthesis 2) is positioned below the renal artery 19.

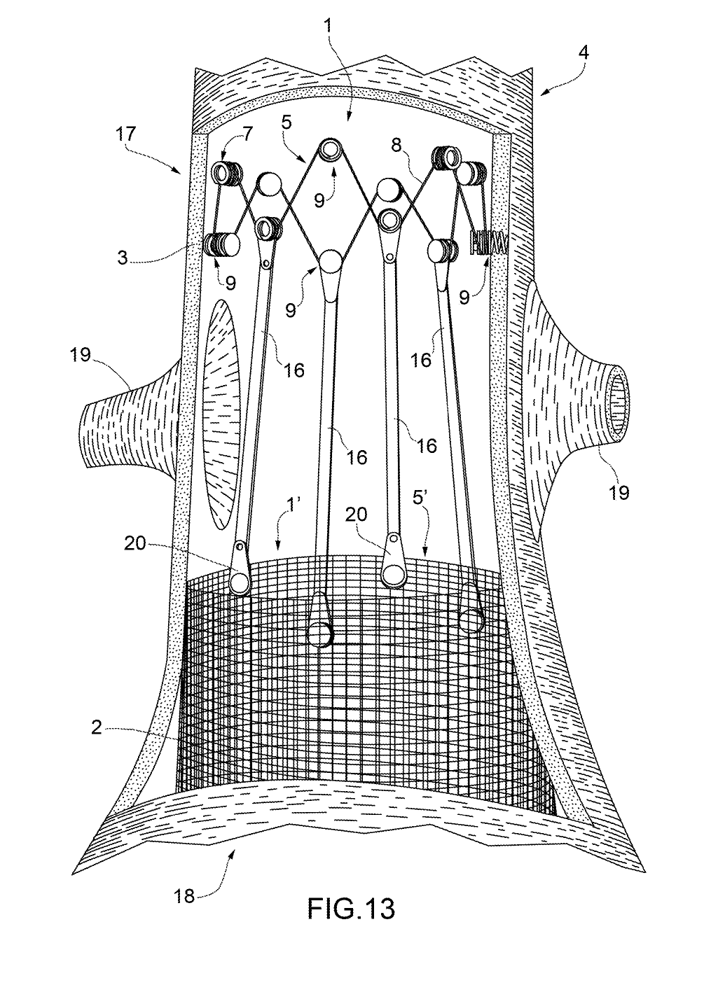

According to the embodiment of FIG. 13, the system 18 comprises (at least) one endoprosthesis 2 and one (only) device 1 for fixing as described above (relative to the first embodiment of the present invention) tied to the endoprosthesis 2. The endoprosthesis 2 and the device 1 are connected by means of two or more (advantageously, at least three; in particular, at least four) connecting elements 16 as described above.

Each connecting element 16 is tied at an end opposite the thrust element 5 to the endoprosthesis 2 by means of a respective coupling 20.

According to a specific use of these embodiments of the system 18, the device 1 is arranged inside the aorta 4 above the renal artery 19 (in a distal position) and the endoprosthesis 2 is positioned below the renal artery 19.

According to a fourth aspect of the present invention, a kit is provided for the repair comprising at least one endoprosthesis 2 and at least one device 1 for fixing as described according to the first embodiment of the present invention.

According to some embodiments, the kit comprises two devices 1 for, fixing as described above connected to one another by means of two or more (advantageously, at least three; in particular, at least four) connecting elements 16 as described above. In these cases, in other words, the kit comprises the system 17 for fixing (as described above), or the components (separate from one another) of the system 17 itself.

According to further embodiments, the kit comprises one single fixing device 1.

The subject matter of the present invention has several advantages and innovative characteristics with respect to the state of the art. These include:
Complete fixing despite anatomical variations.
Transluminal fixing by
   penetration of the endograft;
   penetration into the aortic wall adapting to its configuration;
   withstanding of the pulsatile force of the aorta.
Possibility of use both during the primary implant and subsequently.
Possibility of use in some forms of aortic dissection.
Device available and utilisable in various dimensions.
Possibility of use also in unsupported endograft.
Possibility of offer with a complete kit (with endograft and device).
Possibility of complete remote control.

1. A device to be fixed to a wall of a duct of the human body; the fixing device comprises:
a thrust element, which has the shape of a closed curve so as to peripherally delimit an inner free space and is adapted to exert an outwards spring-back force;
the thrust element comprises a plurality of springs, which are connected to one another; each spring comprises at least one relative loop and is adapted to help the thrust element extend;
the fixing device furthermore comprises a plurality of couplings, which are adapted to fixing the thrust element to the wall of the duct and each comprise a respective fixing element;
each fixing element is adapted to penetrate the wall;
the springs comprise a plurality of torsion springs, each of which has at least one loop delimiting a respective inner lumen, in the area of which a respective coupling is partially arranged;
each coupling comprises a respective support portion, provided with a seat, which houses the loop of the respective spring;
the fixing element being integral with the support portion;
the support portion being structured so as to allow the loop to run inside the seat in such a way as to permit relative rotation of the fixing element with respect to the relative spring.

2. The fixing device according to claim 1, wherein each spring comprises a torsion spring and is connected to two springs, so that the two springs themselves are arranged in sequence.

3. The fixing device according to claim 1, wherein the thrust element comprises a plurality of connecting arms with an elongated shape, in particular an oblong shape; each connecting arm connects two successive springs in an integral manner.

4. The fixing device according to claim 1, wherein each spring comprises two connecting arms with an elongated shape, in particular an oblong shape, and a spring body, which comprises the loop and from whose ends the connecting arms project; the connecting arms of a spring define a free angle ranging from 10° to 180°.

5. The fixing device according to claim 1, wherein the inner free space has an area ranging from 50 mm2 to 30 cm2 when the thrust element is in a completely extended configuration, and up to 600 mm2 when the thrust element is in a packed configuration; possibly, each connecting arm has a length ranging from approximately 5 mm to approximately 5 cm.

6. The fixing device according to claim 1, and comprising at least three couplings; the springs comprising at least three torsion springs.

7. The fixing device according to claim 1, wherein each fixing element is adapted to penetrate the wall rotating around a respective axis; in particular, each fixing element has a longitudinal extension from 0.8 to 2 mm.

8. The fixing device according to claim 1, wherein the fixing element has a shape selected in the group consisting of: substantially helicoidal, substantially spiral, hook-shaped and a combination thereof.

9. The fixing device according to claim 1 and comprising a plurality of, in particular at least three, connecting elements, in particular cables, which are in particular adapted to connect the device to the endoprosthesis.

10. The fixing device according to claim 9, wherein the springs comprise a plurality of, in particular at least three, torsion springs, each of which has at least one loop delimiting an inner lumen; each connecting element is connected to the thrust element in the area of a relative spring.

11. The fixing device according to claim 9, and comprising a further thrust element, which has the shape of a closed curve so as to peripherally delimit a second inner free space and is adapted to exert a second outwards spring-back force; the thrust element and the further thrust element are connected by at least three connecting elements; and wherein the further thrust element of comprises a second plurality of couplings.

12. A system to be fixed to a duct of the human body; the system comprises at least one fixing device according to claim 1 and at least one further fixing device according to claim 1, the at least one further fixing device further comprising at least three cables which are adapted to connect the at least one further fixing device to an endoprosthesis.

13. A system for repairing a duct of the human body comprising at least one endoprosthesis and at least one fixing device according to claim 1; the fixing device and the endoprosthesis being connected to each another.

14. The system according to claim 13, wherein the fixing device is arranged in the area of the end of the endoprosthesis; and the system for repairing a duct comprising a further fixing device according to claim 1, the at least one further fixing device further comprising at least three cables which are adapted to connect the at least one further fixing device to the endoprosthesis, the at least one further fixing device tied to the fixing device by means of the cables.
